(19) United States
(12) Patent Application Publication
(10) Pub. No.: US 2002/0168352 A1
(43) Pub. Date: Nov. 14, 2002

(54) Use of a C1 esterase inhibitor for preventing or delaying rejection of xenotransplants in mammals

(76) Inventors: Michael Winkler, Hemmingen (DE); Horst Rueckoldt, Lehrte (DE)

Correspondence Address: Finnegan, Henderson, Farabow,, Garrett & Dunner, L.L.P., 1300 I Street, N.W., Washington, DC, 20005-3315, US

(21) Appl. No.: 10/094,966

(22) Filed: Mar. 12, 2002

(30) Foreign Application Priority Data
Mar. 14, 2001 (DE) 101 12 617.4

(51) Int. Cl. A61K 38/43; A61K 39/395

(52) U.S. Cl. 424/94.1; 424/146.1

(57) ABSTRACT

The use of a preparation comprising a C1 esterase inhibitor (C1-INH) for preventing or delaying hyperacute and/or acute rejection of xenotransplants in mammals is described. The xenotransplantation is preferably carried out in such a way that a therapeutically effective amount of the C1-INH preparation is administered to the recipient organism before the transplantation and, after the transplantation has taken place, the organism receives daily doses of the C1-INH preparation in an amount sufficient to prevent rejection of the transplant.

The invention relates to the use of a preparation comprising a C1 esterase inhibitor (C1-INH) for improving the prospects of success in xenotransplantation.

Xenotransplantation might, because of the deficit of suitable donor organs, in future achieve considerable importance in surgical technique if it becomes possible to overcome the natural rejection and defense reactions in the event of discordant xenotransplantation. However, at present, hyperacute rejection of xenotransplants (HAR) and acute rejection of xenotransplants (AVR) still stand in the way of permanent success of transplantation of organs from different species of mammals. Neither the immediate rejection caused by preformed antibodies (HAR) nor the rejection which occurs with a delay due to antibodies formed only after the transplantation (AVR) can at present be overcome in xenotransplanation. Nor are immunosuppressive measures such as administration of cyclosporin A, of mycophenolate mofetil or of steroids able to prevent activation of the complement system and cellular damage resulting therefrom. The object therefore is to find a way through permanent inactivation of the complement system which makes sustained compatibility of the xeno-transplant with the recipient organism possible.

The C1 inhibitor, also referred to as C1 esterase inhibitor, is a protein present in blood and is the main inhibitor of the classical pathway of the complement system and the contact system. The C1 inhibitor is able to inhibit the activated form of factor XII and of kallikrein (Schapira M. et al., 1985,

Complement 2: 111; Davis A. E., 1988, Ann Rev Immunol 6: 595; Sim R. B. et al., 1979, FEBS Lett 97: 111; De Agostini A. et al., 1984, J Clin Invest 73: 1542; Fixley R. A. et al., 1985, J Biol Chem 260: 1723; Schapira M. et al., 1982, J Clin Invest 69: 462; Van der Graaf F. et al., 1983, J Clin Invent 71: 149; Harpel P. C. et al., 1975, J Clin Invest 55: 593). The C1 inhibitor thus regulates the activities of two plasma cascades, namely the complement system and the contact system, through which biologically active peptides are produced. The C1 inhibitor is therefore also an important regulator of the inflammatory system. In addition, the C1 inhibitor inhibits activated factor XI (Meijers J. C. M. et al., 1988, Biochemistry 27: 959; Wuillemin W. A. et al., 1995, Blood 85: 1517). As a consequence of this, the C1 inhibitor can be regarded as a coagulation inhibitor. Tissue plasminogen activator and plasmin are also inhibited to a certain extent by the C1 inhibitor, although this is not its main function (Harpel P. C. et al., 1975, J Clin Invest 55: 149; Booth N. A. et al., 1975, Blood 69: 1600).

The C1 inhibitor is obtained to a considerable extent by purification from plasma and is used for clinical applications, in particular in the treatment of hereditary angioedema, a disorder caused by a genetic deficiency of the C1 inhibitor. In addition, it has been reported that good therapeutic results were achieved by administration of the C1 inhibitor for systemic inflammations [International Patent Application WO 92/22320 (Genentech Inc.)], for severe burns, pancreatitis, bone marrow transplantations, administration of the cytokine therapy and on use in extracorporeal blood circulations [DE-A-4 227 762 (Aventis Behring GmbH)].

The complete genomic and the cDNA coding for the C1 inhibitor has already been cloned (Bock S. C. et al., 1986, Biochemistry 25: 4292; Carter P. E. et al., 1988, Eur J Biochem 173: 163). Various variants of the recombinant C1 inhibitor with amino acid mutations in the P1 and the P3 and/or P5 positions of the reactive site, and variants isolated from patients with hereditary angioedema, have already been prepared (Eldering E. et al., 1988, J Biol Chem 263: 11776; Eldering E. et al., 1993, J Biol Chem 267: 7013; Eldering E. et al., 1993, J Clin Invest 91: 1035; International Patent Application WO 91/06650 from Cetus Corporation; Davis A. E. et al., 1992, Nature Genetics 1: 354; Eldering E. et al., 1995, J Biol Chem 270: 2579; Verpy et al., 1995, J Clin Invest 95: 350).

The C1 inhibitor belongs to the large family of serine proteinase inhibitors, which are also called serpins (Travis J. et al., 1983, Ann Rev Biochem 52; 655; Carrel R. W. et al., 1985, Trends Bioch Sci 10: 20). On SDS polyacrylamide gels, the C1 inhibitor shows a molecular weight of about 105 KD. Its plasma concentration is about 270 mg/l (Schapira M. et al., 1985, Complement 2: 111; Nuijens J H et al., 1989, J Clin Invest 84: 443). The C1 inhibitor is a protein whose plasma level may increase up to two-fold in uncomplicated infections and other inflammations (Kalter E S et al., 1985, J. Infect, Dis 151: 1019). Increased formation of C1 inhibitor in inflammations probably serves to protect the body against the harmful effects of the intravascular activation of the complement system and the contact system during the acute reactions.

The serpins act as inhibitors through formation of bimolecular complexes with the proteinase to be inhibited. In these complexes, the active site of the proteinase is bound by the active site of the serpin and thus becomes inactive (Travis J. et al., 1983, Ann Rev Biochem 52: 655). The serpins react specifically with particular proteinases, this specificity being determined by the amino acid sequences of the reactive site.

The present invention is based on the observation that rejection of a xenotransplant can be blocked by administration of a C1 inhibitor.

The invention therefore relates to the use of a preparation comprising a C1 esterase inhibitor (C1-INH) for preventing or delaying hyperacute and/or acute rejection of xenotransplants in mammals, preferably in humans.

A human C1 esterase inhibitor is preferably used on transplantation of an organ of a mammal to a primate. The procedure in this case is for a therapeutically effective amount of the C1-INH preparation to be administered to the recipient organism before the transplantation and, after the transplantation has taken place, for the organism to receive daily doses of the C1-INH preparation in an amount sufficient to prevent rejection of the transplant. Before the transplantation, generally an amount of not more than 150 I.U. of C1-INH per kg of body weight will be administered. After the transplantation has taken place, the amount of C1-INH administered is generally not more than 80 I.U./kg of body weight.

Transfer of a porcine organ to a primate was investigated as a model of transplantation methods of the invention. The skilled worker is also able with the information found to make xenotransplantation possible in humans.

There have already been some attempts to prevent the rejection of xenotransplants. Thus, International Patent Application WO 97/16064 describes a xenotransplantation method in which transgenic tissues or organs are introduced into the human recipient organism, whereby a reduction in the hyperacute rejection reaction (HAR) is observed by comparison with human recipient organisms which receive non-transgenic tissues or organs. There have also been perfusion studies in which it was shown that the survival time of porcine kidneys perfused with fresh human blood containing C1-INH could be extended (22). It was presumed that the alleviation of the hyperacute rejection (HAR) was probably caused by the inhibition of complement. Surprisingly, it is now possible also to apply this method to transplanted organs if, after appropriate pretreatment, a sufficiently high C1-INH dose is ensured continuously in the recipient organism.

The following investigations have been carried out:

Material and Methods

Animals: 5 Cynomolgus Monkeys (Macaca fascicularis)

with a weight of 3.3 to 4.7 kg and an age of 4.5 to 5 years were used in some cases for pharmacokinetic investigations (n=2) or for kidney xenotransplantations (n=3). The monkeys had been purchased from the German primate center in Gottingen and had been tested with negative findings for macacque herpes B (cercopithecine herpes virus type 1), Ebola, Marburg, simian T-cell leukemia virus (STLV), simian retrovirus D (SRV) and simian immunodeficiency virus (SIV).

The donor animals used for the xenotransplantation experiments were 3 large white pigs (Schweinezuchtverband Weser-Ems, Oldenburg, Germany) which had not been pretreated. The pigs were 8 to 18 weeks old and weighed 18 to 23 kg. All the surgical procedures and the postoperative treatment were carried out in agreement with the National German Institute for Animal Care and had been approved by the local animal welfare authorities.

Kidney Xenotransplantation

The removal of the donor organ and the transplantation of the xenokidney with retention of the recipient's own kidneys which were incapable of functioning, and connection thereof to its bladder was carried out by known methods. Closure of the natural urethers with ligatures attached during the operation was checked both by ultrasound or NMR and by autopsy. The spleen was not removed. For postoperative intravenous drug therapy, a connection system was implanted into the right internal jugular vein of all the monkeys.

Definition of HAR and AVR

Xenotransplant HAR meant complete thrombosis of the transplant with vessel connections which showed no bleeding on performance of a biopsy two hours after reperfusion. In addition, signs of HAR [3, 16, 17] in the standard HE sections had to be present both on histological examination and in immunohistochemical investigations. The AVR was diagnosed on the basis of clinical and histological parameters. After all reasons not attributable to the tissue rejection for a deterioration in transplant function such as sepsis or technical errors had been precluded (by ultrasound), any event connected with an increase in the creatinine level of 20% above the baseline was ascribed to AVR.

Immunosuppressant Therapy

The immunosuppression consisted of cyclosporin, mycophenolate mofetil and prednisolone [9, 10, 24]. Cyclosporin was initially given orally one day before the transplantation in a dose of 100 mg/kg. After the transplantation, CyA was administered orally in a dose sufficient to reach a blood level of from 400 to 600 ng/l, at which adequate efficacy and minimal toxicity is reached in this primate species. Administration of MMF by stomach tube started 3 days before the transplantation. The MMF dose was adjusted so that an MPA plasma level of 1 to 4 μg/ml was maintained. The steroids were administered as follows: 1 mg/kg methylprednisolone was given intravenously on the day of the transplantation, followed by oral administration of 1 mg/kg prednisolone in the first week after the transplantation. It was then reduced by 0.05 mg/kg each day in order to reach a maintenance dose of 0.2 mg/kg. Animals No. 3 and No. 4 were also given cyclophosphamide i.v. for induction therapy on days −1, 0, 2 and 4 in an amount of 40 mg/kg, 10 mg/kg and 20 mg/kg respectively. No cyclophosphamide induction therapy was carried out for animal No. 5.

Postoperative Blood Testing

Blood samples were taken each day for determination of the full blood count, the urea, the creatinine and the electrolytes. In addition, 24-hour urine was collected in selected animals and analyzed for the electrolyte concentrations. The cyclosporin levels in the whole blood, as well as the MPA plasma levels were determined by measurement methods using monoclonal antibodies (EMIT 2000, Aventis Behring GmbH).

Flow Cytometry

Preoperative and postoperative sera were assayed for their antiporcine antibody titers using a flow cytometry assay for analysis. After taking blood, the serum was prepared by centrifugation at 3 400×g for 10 minutes at 20° C. To detect antiporcine antibodies, frozen aliquots of porcine peripheral blood leukocytes (PPBL) which had been obtained from an individual large white pig were thawed, and 0.5×10⁵ cells were stained by 20 μl of the corresponding cynomolgus serum in various solutions. After incubation at 4° C. for 20 minutes, the cells were washed twice with PBS which contained 1% BSA and 0.1% sodium azide. Bound cynomolgus antibodies were detected using goat anti-human FITC secondary antibodies which reacted with IgG (Dianova, Hamburg) or IgM (Dianova, Hamburg). These antibodies are known to cross-react with cynomolgus immunoglobulins [7]. The antibodies had been preabsorbed with porcine serum. After incubation at 4° C. for 20 minutes, the cells were again washed twice and then analyzed in a FACScan (Becton Dickinson, Mountain View, Calif.) cytometer.

Analysis of the Complement Levels

To detect the plasma complement levels, venous blood was taken and EDTA was added as anticoagulant. Immediately after taking the blood, the samples were centrifuged; the plasma was then stored at −70° C. until analyzed. The concentrations of C3a and sC5b-9 in the cynomolgus plasma were measured using commercially available ELISA reagents (Quidel, San Diego, Calif., USA) which are known to cross-react with cynomolgus complement compounds. These investigations were carried out exactly in accordance with the manufacturers' instructions.

Analysis of the C1-INH Activity

Both the total C1-INH activity and the total amount of the C1-INH protein in citrated plasma was measured quantitatively. The activity of C1-INH was measured as follows: C1-INH in the sample inhibits a defined volume of exogenous C1 esterase. The remaining activity of the C1 esterase is measured in a kinetic assay by measuring the increase in the absorption at 405 nm (Berichrom® C1-Inhibitor, Dade Behring Inc., Newark, USA). The C1 activity of the samples was calculated from a reference plot produced from human standard plasma. Normal values (for humans) were defined according to a standard which had been supplied by the manufacturer of the C1-INH assay. The amount of C1-INH protein was determined by NOR-Partigen, Behring, using anti-C1-INH antibodies from sheep and from rabbits. The investigation was carried out exactly in accordance with the manufacturer's instructions. In order to compare the extent of complement activation after reperfusion of the transplant, use was also made of historical data from two groups of monkeys which had received an unmodified or an h-DAF transgenic transplant but no supplementary C1-INH treatment [8]. The immediate postoperative immunosuppression in these animals likewise consisted of CyP, CyA and low doses of steroids, but no mycophenolate mofetil was given to these animals.

Results

For the pharmacokinetic analysis, a single i.v. dose of C1-INH was given to two healthy cynomolgus monkeys (experimental animals No. 1 and No. 2). Based on experience with C1-INH administration in humans [26, 27], a dose of 150 I.U./kg was chosen. The pharmacokinetic data showed a concentration of more than 200% of the normal value. The calculated half-life was 72 and 90 hours respectively. Accordingly, a dose of 500 I.U. of C1-INH, which was given 1 hour after the reperfusion, and 80 I.U./kg a day thereafter was used in the following kidney xenotransplantations on monkeys.

A life-supporting porcine kidney xenotransplantation was performed in three monkeys. The postoperative immunosuppression comprised CyA, MMF and steroids. A CyP induction was additionally administered in animals No. 3 and No. 4 in compliance with a protocol previously established in another laboratory [25]. Animal No. 5 was given no CyP induction. C1-INH (250 I.U.) was additionally administered each day after the transplantation to all three monkeys. The systemic C1 values after the transplantation were in the range from 170 to 210% of the normal values. There was a remarkably small variation in the total C1-INH levels between the individuals, especially during the first 5 days after the operation.

None of these three xenotransplants underwent hyperacute rejection. All three porcine kidneys showed urine production initially; a stable kidney function was produced in the postoperative course, minimal amounts of creatinine of, respectively, 31, 71 and 64 μmol/l being found. In all three animals, analysis of the systemic antiporcine antibody levels by FACS showed no significant increases in the titer during the observation period.

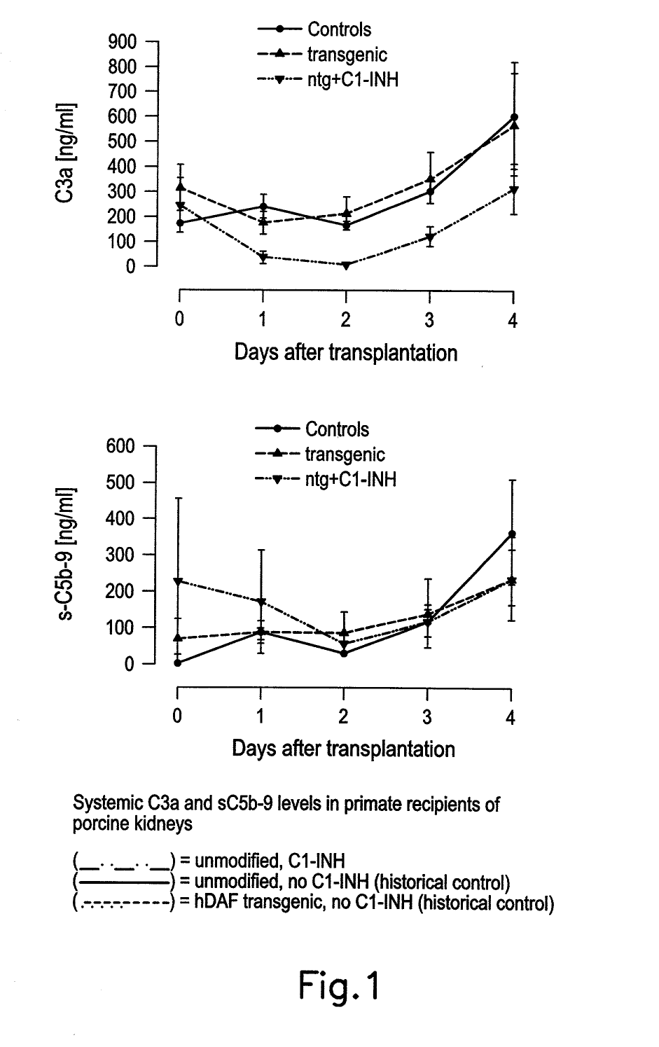

The plasma levels of C3a and sC5b-9 were measured in the three monkeys by ELISA. These data were compared with a set of historical data found in monkeys after an unmodified (n=3) or hDAF transgenic (n=10) kidney transplantation without continuous administration of C1-INH therapy (FIG. 1). Whereas the sC5b-9 levels were comparable in all the groups, the C3a levels tended to have lower values in the C1-INH group compared with the two other groups. All three monkeys were lost on days 6, 13 and 15 after the transplantation because of bacterial sepsis while the kidney function was stable (urine production, serum creatinine level). The sepsis did not respond to anti-microbial treatment. In all three monkeys, positive blood cultures were found for E. coli/E. faecalis (animal No. 3), Streptococcus ssp. (animal No. 4) and Staphylococcus aureus (animal No. 5). Whereas stable kidney function was found in all three monkeys after the transplantation, there was a slight rise in the creatinine level in animals No. 3 and No. 4. Impairments of the coagulation system were found in all the monkeys, with an increase in the partial thromboplastin time, increased fibrinogen degradation products and reduced AT-III concentrations. In one animal (animal No. 5), clinical signs of DIC were visible in all organ systems at autopsy, whereas there were no signs of clinically manifest coagulation disturbances found in the other two animals—despite a considerable increase in the fibrinogen degradation products.

List of References:

1. White D J G, Oglesby T, Liszewski M K, et al. Expression of human decay accelerating factor or membrane cofactor protein genes on mouse cells inhibits lysis by human complement. Transplant Proc 1992: 24: 474.

2. Cozzi E, White D. The generation of transgenic pigs as potential organ donors for humans. Nature Med 1995: 1: 964.

3. Zaidi A, Schmoeckel M, Bhatti F, et al. Life-supporting pig-to-primate renal xenotransplantation using genetically modified donors. Transplantation 1998: 65: 1584.

4. Caine R Y. Organ Transplantation between widely disparate species. Transplant Proc 1970: 2: 550.

5. Sablinski T, Gianeilo P R, Bailin M, et al. Pig to monkey bone marrow and kidney xenotransplantation. Surgery 1997: 121: 381.

6. Platt J L, Lin S S, McGregor C G. Acute vascular rejection. Xenotransplantation 1998: 5: 169.

7. Loss M, Kunz R, Przemeck M, et al. Influence of cold ischemia time, pretransplant anti-porcine antibodies and donor/recipient size matching on hyperacute graft rejection following discordant porcine to cynomolgus kidney transplantation. Transplantation 2000; 69: 1155.

8. Loss M, Vangerow B, Schmidtko J, et al.: Acute vascular rejection is associated with systemic complement activation in a pig-to-primate kidney xenograft model. Xenotransplantation 2000; 7: 186.

9. Davis E A, Pruitt S K, Greene P S, et al. Inhibition of complement, evoked antibody, and cellular response prevents rejection of pig-to-primate cardiac xenografts. Transplantation 1996: 62: 1018

10. Waterworth P D, Cozzi E, Tolan M J, et al. Pig-to-primate cardiac xenotransplantation and cyclophosphamide therapy. Transplant Proc 1997: 29: 899.

11. Fraiser L H, Kanekal S, Kehrer J P, Cyclophosphamide toxicity: characterizing and avoiding the problem. Drugs 1991: 42: 781.

12. Besse T, Duck L, Latinne D, et al. Effect of plasmapheresis and splenectomy on parameters involved in vascular rejection of discordant xenografts in the swine to baboon model. Transplant Proc 1994: 26: 1042.

13. Leventhal J R, Removal of natural antibodies by immunoabsorption: Results of experimental studies. In: Cooper D K C, Kemp E, Platt J L, White D J G, eds. Xenotransplantation: the Transplantation of organs and tissues between species. Berlin: Springer-Verlag, 1997.

14. Miyatake T, Sato K, Takigami K, et al. Complement-fixing elicited antibodies are a major component in the pathogenesis of xenograft rejection. J Immunol 1998: 160: 4114.

15. Sato K, Takigami K, Miyatake T, et al. Suppression of delayed xenograft rejection by specific depletion of elicited antibodies in the IgM isotype. Transplantation 1999: 68: 844.

16. Platt J L, Fischei R J, Matas A J, et al. Immunopathology of hyperacute xenograft rejection in a swine-to-primate model. Transplantation 1991: 52: 214.

17. Dalmasso, A. P., Vercellotti, G. M., Fischel, R. J. et al., Mechanism of complement activation in the hyperacute rejection of porcine organs transplanted into primate recipients. AM J. Pathol 1991: 140: 1157.

18. Lawson J H, Platt J L. Molecular barriers to xenotransplantation. Transplantation 1996: 62: 303.

19. McCurry K R, Parker W, Cotterell A H, et al. Humoral responses to pig-to-baboon cardiac transplantation: implications for the pathogenesis and treatment of acute vascular rejection and for accommodation. Hum Immunol 1997: 58: 91.

20. Dalmasso A P, Platt J L. Prevention of complement-mediated activation of xenogenic endothelial cells in an in vitro model of xenograft hyperacute rejection by C1 inhibitor. Transplantation 1993: 56: 1171.

21. Heckl-Ostreicher B, Wosnik A. Kirschfink M. Protection of porcine endothelial cells from complement regulators CD59, C1 inhibitor, and soluble complement receptor type 1. Analysis in a pig-to-human in vitro model relevant to hyperacute xenograft rejection. Transplantation 1996: 62: 1693.

22. Fiane A E, Videm V, Johansen H T, et al. C1-INHibitor attenuates hyperacute rejection and inhibits complement, leukocyte and platelet activation in an ex vivo pig-to-human perfusion model. Immunopharmacology 1999: 42: 231.

23. Fodor W., Williams B., Matis L.: Expression of a functional human complement inhibitor in a transgenic pig as a model for the prevention of xenogenic hyperacute organ rejection. Proc. Natl. Acad. Sci. USA. 1994; 91:1 1 153

24. Valdivia L. A., Murase N., Rao A. S., et al.: Use of Tacrolimus (FK 506) and Antimetabolites as Immuno-suppressants for Xenotransplantation across closely related rodent species. In: Cooper, Kemp, Platt, White (eds.): Xenotransplantation. The Transplantation of Organs and Tissues between Species. Springer, Bedin, 1996: 328.

25. Soin B, Ostlie D, Cozzi E, Smith K G, Bradley J R, Vial C, Masroor S, Lancaster R, White D J, Friend P J: Growth of porcine kidneys in their native and xenograft environment. Xenotransplantation 2000 May; 7 (2): 96-1 00

26. Radke A, Mottaghy K, Goldmann C, Khorram-Sefat R, Kovacs B, Janssen A, Klosterhalfen B, Hafemann B, Pallua N, Kirschfink M: C1 inhibitor prevents capillary leakage after thermal trauma. Crit Care Med 2000 September; 28(9): 3224-32

27. G. Marx, B. Nashan, M. Cobas Meyer, B. Van-gerow, H. J. Schlitt, S. Ziesing, M. Leuwer, S. Piepenbrock, H. Rueckoldt: Septic shock after liver Transplantation for Caroli's disease: clinical improvement after treatment with C1-esterase inhibitor. Intensive Care Med 1999, 25: 1017

28. B. Vangerow, J. Hecker, R. Lorenz, M. Loss, M.Przewmeck, R. Appiah, J. Schmidtko, A. Jalali H. Rueckoldt, M. Winkler: C1-Inh for treatment of acute vascular xenograft rejection in cynomolgus recipients of HDAF transgenic porcine kidneys. Xenotransplantation (in press).

1. The use of a preparation comprising a C1 esterase inhibitor (C1-INH) for preventing or delaying hyperacute and/or acute rejection of xenotransplants in mammals.

2. The use as claimed in claim 1, wherein the preparation comprises human C1 esterase inhibitor.

3. The use as claimed in claims 1 and 2, wherein a therapeutically effective amount of the C1-INH preparation is administered to the recipient organism before the transplantation and, after the transplantation has taken place, the organism receives daily doses of the C1-INH preparation in an amount sufficient to prevent rejection of the transplant.

4. The use as claimed in claims 1 to 3, wherein the amount of C1-INH administered before the transplantation is not more than 150 I.U./kg of body weight.

5. The use as claimed in claims 1 to 4, wherein the amount of C1-INH administered after the transplantation has taken place is not more than 80 I.U./kg of body weight.

6. The use as claimed in claims 1 to 5, wherein the xenotransplant is of porcine origin and the recipient is a primate.

7. The use as claimed in claim 6, wherein the recipient is a human.
